(19) United States
(12) Patent Application Publication
(10) Pub. No.: US 2005/0288568 A1
(43) Pub. Date: Dec. 29, 2005

(54) Real-time automatic searching system for medical image and method for the same

(76) Inventor: William Pan, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/873,247

(22) Filed: Jun. 23, 2004

(51) Int. Cl. A61B 5/05

(52) U.S. Cl. 600/407

(57) ABSTRACT

A real-time automatic searching system for medical image in a hospital and a method for the same are proposed. The system comprises at least one medical instrument to generate a digital image for a body portion of a patient; a remote host connected to the medical instrument through a network and storing a plurality of basic data for patient, a plurality of digital images for patient and a plurality of reference images; at least one terminal computer at a doctor office and connected to the remote host though the network. The terminal computer has an auto searching routine executable thereon for automatically fetching a basic data of a patient under diagnosis, searching the remote host for reference image with associated gender, age and body portion to the patient, and displaying a searched reference image on the terminal computer.

### BACKGROUND OF THE INVENTION

1. Field of the Invention

The present invention relates to a real-time automatic searching system for medical image and method for the same, especially to a real-time automatic searching system with which doctor in hospital can easily compare a local image of patient with normal reference image for diagnosis and method for the same.

2. Description of Related Art

As the progress of computer technology, computer system is now employed in hospital for regulating patient register, doctor prescription, hospital faculty and patient administration etc.

Moreover, computer technology also provides valuable help to medial instruments. For example, 3D computer imaging technology can be used in ultrasonic imaging instruments for depicting internal image of patient or figure of fetus. The computer technology can also be applied to tomography and endoscopes instruments to digitalize the taken images thereof. The digital image is helpful diagnosis aid for doctor.

In modern hospital, the doctors often survey an examination report for patient before their diagnosis. The examination report generally contains medical image plates obtained by X-ray scanning, tomography scanning or ultrasonic scanning.

The conventional medical image plates generally have not uniform size. Moreover, the conventional image plates are not physically attached to the examination report because they lack of flexibility. Therefore, it is difficult to integrate the resource of medical image plates and examination report. To overcome this drawback, a DICOM (Digital Imaging Communication in Medicine) format is proposed to digitalize medical image plates such that the digitalized medical images can be easily stored in computer and shared by others.

FIG. 1 is a schematic diagram of a diagnostic window for a computer-aided diagnostic system in a hospital. The personal data, medical record of a patient can be displayed on the diagnostic window together with the digitalized medical images.

However, a doctor often needs a normal reference image to compare it with the medical image of a patient in order to diagnose the patient correctly. For example, to diagnose a suspected SARS patient, the doctor needs an image of normal lung for comparison with the lung image of the patient.

### SUMMARY OF THE INVENTION

It is the object of the present invention to provide a real-time automatic searching system for medical image in a hospital and a method for the same. By this system and method, the digital image of a body portion of a patient and the basic data of patient can be displayed on a terminal computer at local doctor side. The reference images for healthy people and images for patient with specific disease are searched with regard to the gender, age and body portion of the patient under diagnosis. The reference images are displayed on the terminal computer for easy comparison of doctor.

To achieve the above object, the present invention provides a real-time automatic searching method for medical image in a hospital, comprising the steps of providing a reference image database storing a plurality of reference images; generating a digital image for a body portion of a patient by using a medical instrument and storing the digital image in a patient database with a basic data of the patient; searching the reference image database to find at least one matched reference image associated with people having corresponding gender, age and body portion with the patient; displaying the matched reference image and the digital image on a terminal computer.

To achieve the above object, the present invention provides a real-time automatic searching system for medical image in a hospital. The system comprises at least one medical instrument to generate a digital image for a body portion of a patient; a remote host connected to the medical instrument through a network and storing a plurality of basic data for patient, a plurality of digital images for patient and a plurality of reference images; at least one terminal computer at a doctor office and connected to the remote host though the network. The terminal computer has an auto searching routine executable thereon for automatically fetching a basic data of a patient under diagnosis, searching the remote host for reference image with associated gender, age and body portion to the patient, and displaying a searched reference image on the terminal computer.

The above summaries are intended to illustrate exemplary embodiments of the invention, which will be best understood in conjunction with the detailed description to follow, and are not intended to limit the scope of the appended claims.

### BRIEF DESCRIPTION OF THE DRAWINGS

The features of the invention believed to be novel are set forth with particularity in the appended claims. The invention itself however may be best understood by reference to the following detailed description of the invention, which describes certain exemplary embodiments of the invention, taken in conjunction with the accompanying drawings in which:

FIG. 1 is a schematic diagram of a diagnostic window for a computer-aided diagnostic system in a hospital.

FIG. 2 shows a schematic view of real-time automatic searching system for medical image according to the present invention.

FIG. 3 shows a schematic view of first preferred embodiment of the present invention.

FIG. 4 shows a schematic view of second preferred embodiment of the present invention

FIGS. 5A and 5B show flowchart according to the present invention.

FIG. 6 shows a schematic view of the menu window.

### DETAILED DESCRIPTION OF THE PREFERRED EMBODIMENTS

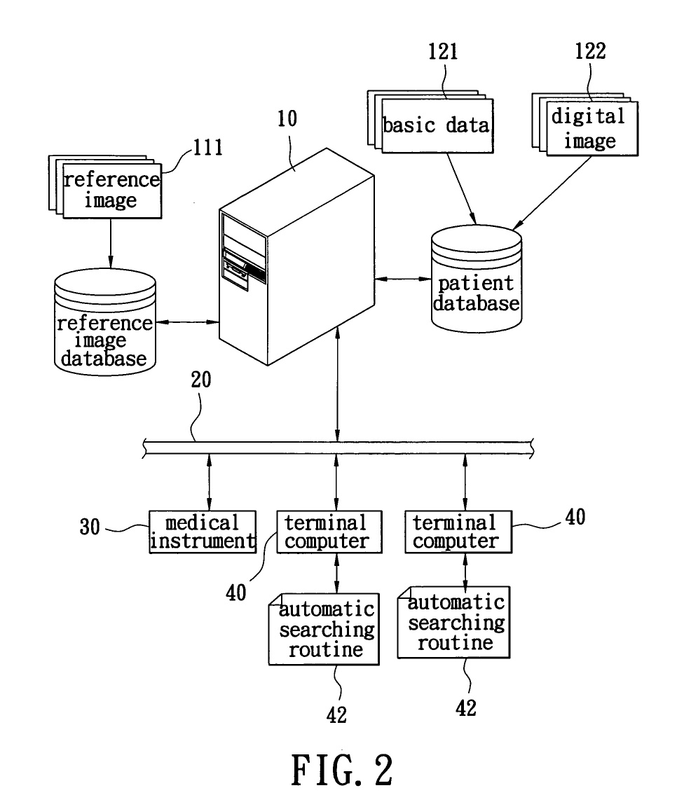

FIG. 2 shows a schematic view of real-time automatic searching system for medical image according to the present invention. The real-time automatic searching system for medical image is employed in a medical intuition such as hospital to provide doctor with real-time medical image. The real-time automatic searching system according to the present invention comprises a remote host 10, at least one medical instrument 30 in a hospital and a plurality of terminal computers 40 in doctor office, wherein the remote host 10 is connected to the medical instrument 30 and the terminal computers 40 through a network 20 such as a local area network, Internet or wireless network.

The remote host 10 comprises a reference image database 11 and a patient database 12. The reference image database 11 stores a plurality of reference images including images for healthy people and images for patient with specific disease. Moreover, the reference images in the reference image database 11 are classified with reference to gender, age and body portion. The patient database 12 stores basic data 121 of patient and digital image 122 of patient, wherein the basic data 121 includes medical record number, name, age, gender and body portion of patient.

The digital image 122 of patient is fetched by the medical instrument 30, wherein the digital image 122 is related to a body portion of the patient and stored in the patient database 12 with the basic data 121. The medical instrument 30 is compatible with DICOM format and equipped with DICOM console to generate digital image 122 with DICOM format. The medical instrument 30 can be, for example, an X-ray imager, an angiography camera, an interiorly camera or a tomography imager.

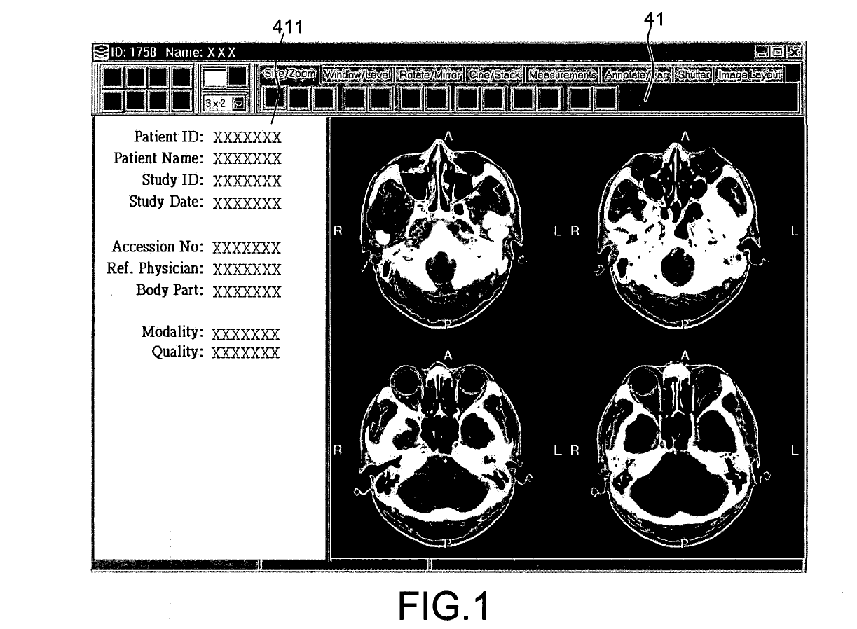

The terminal computer 40 in doctor office can display the basic data 121 of patient and digital image 122 of patient by connecting to the remote host 10 through the network 20. FIG. 1 shows the diagnostic window 41 on the terminal computer 40. The diagnostic window 41 includes a basic data area 411 for displaying the basic data 121 of patient and a patient image area 412 for displaying the medical digital image 122 of patient.

The terminal computer 40 includes an automatic searching routine 42 thereon; the automatic searching routine 42 can automatically fetch the basic data 121 of patient and search a reference image 111 in the reference image database 11, which is an image of normal people with related gender, age and body portion with the patient under test, or an image of people with similar pathology to and related gender, age and body portion with the patient under test. The reference image 111 is also shown on the terminal computer 40 for doctor to compare it with the digital image 122 of patient and make a diagnosis.

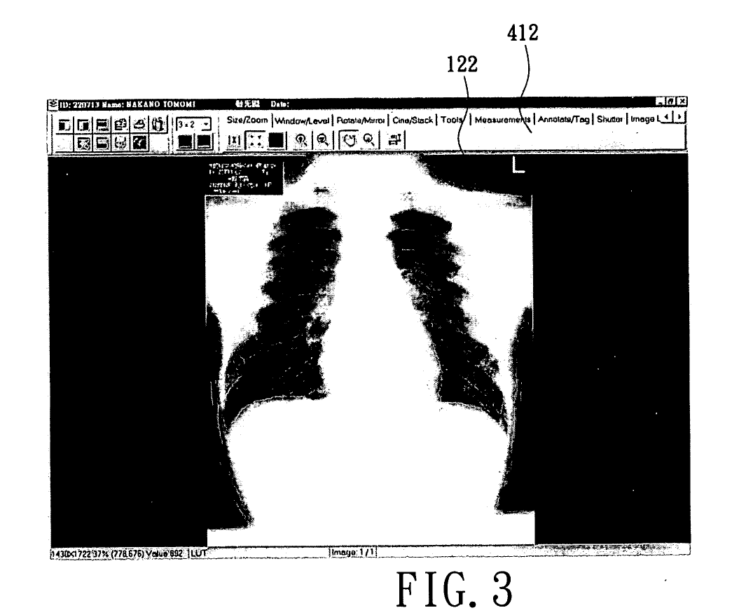

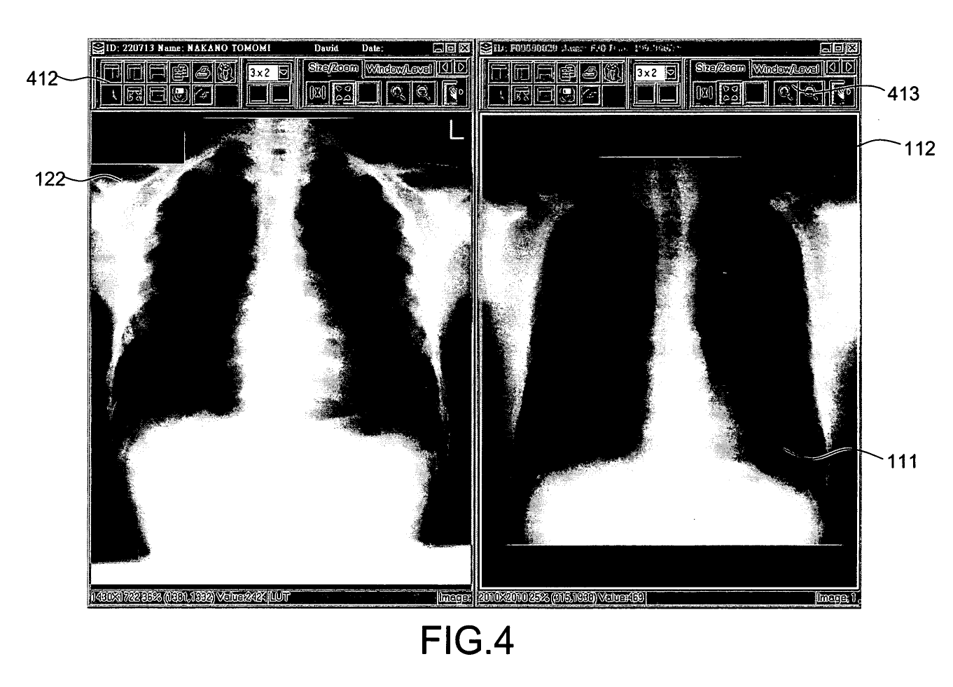

FIG. 3 shows a schematic view of first preferred embodiment of the present invention, wherein only the digital image 122 of patient is enlarged on the diagnostic window 41. FIG. 4 shows a schematic view of second preferred embodiment of the present invention, wherein a comparison-image inset 413 is also provided in the diagnostic window 41 to display a found reference image 111 and the diagnostic window 41 is operated in a comparison mode. The reference image 111 shown in FIG. 2 is also enlarged to provide better view and is surrounded by highlighted frame to prevent the doctor from confusing it with the digital image 122.

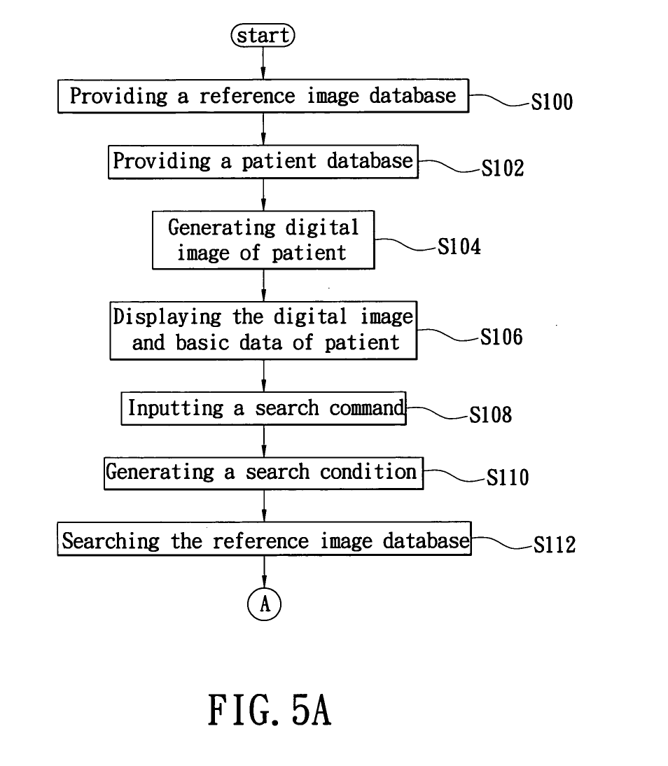

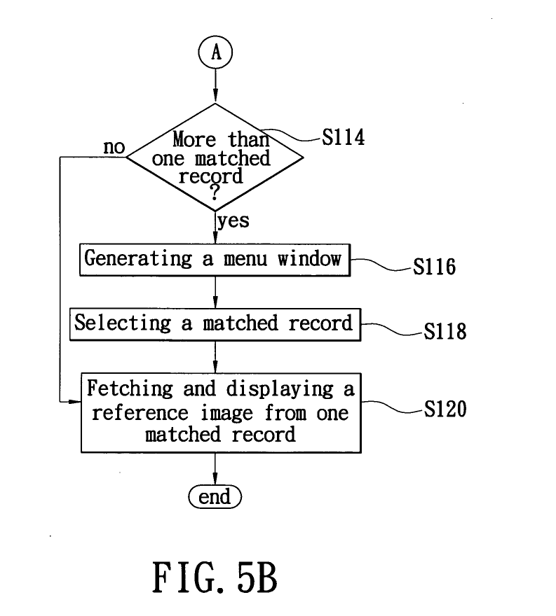

FIGS. 5A and 5B show flowchart according to the present invention. At step S100, a reference image database 11 is provided in the remote host 10 and stores with reference images 111, which are related image of normal people or people with specific disease. At step S102, a patient database 12 is also provided to store basic data 121 of patient and digital image 122 of patient.

Before the diagnosis of doctor, a portion of the patient under diagnosis is examined by a medical instrument 30 to produce at least one digital image 122 of the patient, and the digital image 122 of the patient is stored in the patient database 12 with the basic data 121 of patient at step S104.

During the diagnosis of doctor, the digital image 122 of patient and the basic data 121 of patient are displayed on the terminal computer 40 at step S106 as shown in FIG. 1. Moreover, the doctor can enlarge the digital image 122 for more careful examination as shown in FIG. 3.

If the doctor has difficulty in diagnosis, he (she) can input a search command to the terminal computer 40 at sep S108. The terminal computer 40 generates a search condition in regard with gender, age and body portion of the patient and sends the search condition to the remote host 10. The remote host 10 is then connected to the reference image database 11 for searching the reference image database 11 at step S112, wherein the images for healthy people and images for patient with specific disease are searched according to the search condition, namely, image for people with similar gender, age and body portion etc.

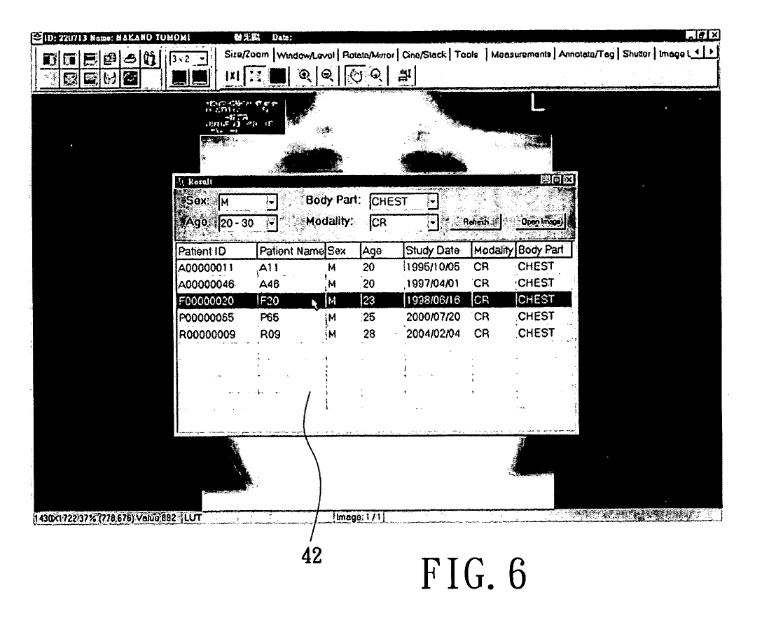

When more than one matched reference images 111 is founded at step S114, the matched reference images 111 is sent to the terminal computer 40 and a menu window 43 is generated at step S116 for displaying all matched reference images 111 (matched records). FIG. 6 shows a schematic view of the menu window 43.

At this time, the doctor can select a matched record from the menu window 43 at step S118. After selection, the remote host 10 fetches the associated reference images 111 and displays the reference image on the terminal computer 40 at step S120. As shown in FIG. 4, both the digital image 122 of patient and the reference images 111 are displayed for facilitating the comparison by doctor.

To sum up, the real-time automatic searching system enables doctor in hospital to easily compare a local image of patient with normal reference image for diagnosis.

Although the present invention has been described with reference to the preferred embodiment thereof, it will be understood that the invention is not limited to the details thereof. Various substitutions and modifications have suggested in the foregoing description, and other will occur to those of ordinary skill in the art. Therefore, all such substitutions and modifications are intended to be embraced within the scope of the invention as defined in the appended claims.

1. A real-time automatic searching method for medical image in a hospital, comprising the steps of
providing a reference image database storing a plurality of reference images;
generating a digital image for a body portion of a patient by using a medical instrument and storing the digital image in a patient database with a basic data of the patient;
searching the reference image database to find at least one matched reference image associated with people having corresponding gender, age and body portion with the patient;
displaying the matched reference image and the digital image on a terminal computer.

2. The real-time automatic searching method as in claim 1, wherein in the step of providing a reference image database, the reference image database stores a plurality of reference image for healthy people and images for patient with specific disease, the reference images being classified with reference to gender, age and body portion.

3. The real-time automatic searching method as in claim 1, further comprising a step before the step of generating a digital image for a body portion of a patient, the step providing a patient database storing the digital image and the basic data of the patient.

4. The real-time automatic searching method as in claim 1, wherein the medical instrument is compatible with DICOM format and equipped with a DICOM console to generate digital image with DICOM format.

5. The real-time automatic searching method as in claim 4, wherein the medical instrument is one of an X-ray imager, an angiography camera, an interiorly camera and a tomography imager.

6. The real-time automatic searching method as in claim 1, further comprising a step after the step of generating a digital image for a body portion of a patient, the step displaying the digital image and the basic data of the patient on a terminal computer.

7. The real-time automatic searching method as in claim 1, further comprising a step before the step of searching the reference image database, the step inputting a searching command to the terminal computer.

8. The real-time automatic searching method as in claim 1, further comprising a plurality of sub-steps in the step of searching the reference image database, the sub-steps comprising
generating a search condition associated with a gender, an age data and a body portion of the patient; and
connecting to the reference image data for searching.

9. The real-time automatic searching method as in claim 8, further comprising steps of:
generating a menu window on the terminal computer for displaying all matched records when more than one matched record is found;
selecting one matched records;
fetching a reference image corresponding to the selected and matched record.

10. A real-time automatic searching system for medical image in a hospital, comprising:
at least one medical instrument to generate a digital image for a body portion of a patient;
a remote host connected to the medical instrument through a network and storing a plurality of basic data for patient, a plurality of digital images for patient and a plurality of reference images;
at least one terminal computer at a doctor office and connected to the remote host though the network to display the basic data for patient and the digital image for patient; and
an auto searching routine executable on the terminal computer for automatically fetching a basic data of a patient under diagnosis, searching the remote host for reference image with associated gender, age and body portion to the patient under diagnosis, and displaying a searched reference image on the terminal computer.

11. The real-time automatic searching system as in claim 10, wherein the medical instrument is compatible with DICOM format and equipped with a DICOM console to generate digital image with DICOM format.

12. The real-time automatic searching system as in claim 11, wherein the medical instrument is one of an X-ray imager, an angiography camera, an interiorly camera and a tomography imager.

13. The real-time automatic searching system as in claim 10, wherein the network is one of a local area network, an Internet and a wireless network.

14. The real-time automatic searching system as in claim 10, wherein the remote host comprises
a patient database for storing a plurality of basic data for patient and a plurality of digital image for patient; and
a reference image database for storing a plurality of reference images.

15. The real-time automatic searching system as in claim 14, wherein the reference image database stores a plurality of reference image for healthy people and images for patient with specific disease, the reference images being classified with reference to gender, age and body portion.

16. The real-time automatic searching system as in claim 10, wherein the terminal computer has a diagnosis window including
a basic data area for displaying the basic data of patient, the basic data including medical record number, name, age, gender and body portion of patient;
a patient image area for displaying the medical digital image of patient; and
a reference image area for displaying the reference image.

17. The real-time automatic searching system as in claim 16, wherein the reference image area is surrounded by a highlighted frame to differentiate with the patient image area.
